(19) United States
(12) Patent Application Publication
(10) Pub. No.: US 2026/0199628 A1
(43) Pub. Date: Jul. 16, 2026

(54) SYSTEMS, METHODS, AND APPARATUS FOR PRODUCING NITRIC OXIDE

(71) Applicant: VERO Biotech Inc., Atlanta, GA (US)

(72) Inventor: Christopher Varga, Atlanta, GA (US)

(21) Appl. No.: 19/444,666

(22) Filed: Jan. 9, 2026

Related U.S. Application Data
(60) Provisional application No. 63/743,731, filed on Jan. 10, 2025.

(51) Int. Cl. A61M 16/12; B01J 8/02; C01B 21/24

(52) U.S. Cl. CPC A61M 16/12; B01J 8/02; C01B 21/24; A61M 2202/0275; A61M 2205/505; A61M 2205/587; A61M 2205/6081

(57) ABSTRACT

Disclosed herein is an apparatus comprising: one or more receptacles, each configured to receive a cassette comprising a nitric oxide-forming media; and a user interface, wherein at least a portion of the user interface is configured to indicate: (a) a status of the one or more receptacles; and/or (b) a status of the cassette if inserted into the one or more receptacle.

### CROSS-REFERENCE TO RELATED APPLICATIONS

This application claims priority to and the benefit of U.S. Provisional Application No. 63/743,731, filed January 10, 2025, the content of which is incorporated herein by reference in its entirety.

### FIELD

Some aspects described herein relate to a medical device for producing and delivering a gas that includes nitric oxide, with the potential to significantly improve patient care.

### BACKGROUND

Some aspects described herein relate to the production of nitric oxide (NO), which is then typically delivered to a patient in a medical setting.

Nitric oxide is a vasodilator indicated to improve oxygenation and reduce the need for extracorporeal membrane oxygenation, particularly in term and near-term neonates with hypoxic respiratory failure associated with clinical or echocardiographic evidence of pulmonary hypertension in conjunction with ventilatory support. Low concentrations of inhaled nitric oxide can also prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, hyaline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism, and idiopathic or primary pulmonary hypertension or chronic hypoxia.

Inhaled nitric oxide therapy typically involves the delivery of nitric oxide in parts per billion to parts per million concentrations within a breathing gas, generally composing air or oxygen-enriched air. This breathing gas may contain other components, such as anesthetic agents, nebulized liquids, or other gaseous components, and it is typically conveyed to a patient using either a mechanical or manual ventilation device. In some inhaled nitric oxide delivery systems, the nitric oxide is provided within pressurized tanks, whereas in other systems, the nitric oxide may be generated on demand within the delivery system itself. One such system is described in US 11,744,978, the content of which is incorporated herein in its entirety. In this approach, nitric oxide is produced by a chemical reaction between NO₂ gas and an antioxidant wherein the NO₂ gas is made through a phase-change of liquid N₂O₄. In such systems, the liquid N₂O₄ is typically housed in a pressure vessel with components required for reaction control (e.g., heating and cooling components), reactant mixing, and measurement co-located with the reactants themselves.

The nitric oxide produced for delivery to the patient is carried in a gas such as air, with the combined air + NO gas flow controlled through a series of fluidic components such as pumps, valves, restrictions, filters, and meters for precise delivery to the patient. In order to initiate the creation of pure nitric oxide from the cassette, there is an activation step that typically involves a mechanical and/or thermal action that promotes the conversion of liquid N₂O₄ to gaseous NO₂ and additionally exposure of NO₂ to incoming air and subsequently to the antioxidant containing media. A user, such as a respiratory therapist, will typically insert a cassette into a console to start the process. The console must then engage the cassette to enable the mechanical and/or thermal actions that begin the chemical process. The actual mechanical and thermal actions are typically caused to occur through the user setting a nitric oxide dose on a user interface through a button press or something similar. Because this must be a sealed process that the user is kept shielded from, there will also be a locking mechanism that prevents the user from removing the cassette while the chemical reaction is proceeding. Once nitric oxide treatment is complete, the user will terminate dosing with another input on the user interface. This will typically initiate another mechanical and/or thermal action that causes the remaining chemical reactants to be inerted to make the cassette safe for removal by the user.

Further to the different actions on a single cassette, the nitric oxide delivery device (e.g., console) that holds the cassette may also contain one or more additional cassettes. In this case, nitric oxide dosing may be achieved through either cassette, or when one cassette nears depletion of its chemical reactants, the system may automatically transition to dosing from one or more additional cassettes. In this way, dosing can be maintained, and the user can be prompted to replace the depleted cassette so that a new cassette is available for dose transfer when the dosing cassette nears depletion. Because of all the various states that the cassette may be in and transition through, there is a desire for a user to be able to visualize the status of a cassette. Current systems may provide some indications of cassette status on a graphical user interface but there is no dosing indication local to the cassette itself.

Accordingly, there is a need for a solution that provides visual feedback of nitric oxide cassette status to the user during various stages of operation. This is especially crucial in a failure of a graphical user interface or computer failure. The present invention provides a novel solution for dosing and cassette status in nitric oxide therapy.

### SUMMARY

The present disclosure is directed to an apparatus comprising: one or more receptacles, each configured to receive a cassette comprising a nitric oxide-forming media; and a user interface, wherein at least a portion of the user interface is configured to indicate: (a) a status of the one or more receptacles; and/or (b) a status of the cassette if inserted into the one or more receptacle.

In still further aspects, the at least a portion of the user interface further comprises one or more elements serving as an indicator of the status of the receptacle and/or the status of the cassette.

In still further aspects, the one or more elements indicate one or more of the statuses of the cassette: inserted, locked, activated, warming, pressuring, ready for dosing, actively dosing, dosing paused, dosing transitioning, dosing complete, cassette in need of replacement, cassette full, cassette empty, cassette near empty, cassette dose remaining, cassette time remaining, cassette failure, cassette error, cassette is not inserted property, cassette ready for ejection, or any combination thereof.

In still further aspects, the one or more elements comprise a plurality of segments, wherein a sequence of the plurality of segments is configured to indicate the status of the cassette. In such exemplary and unlimiting aspects, the sequence and a number of segments indicate an amount of nitric oxide dosing remaining in the cassette, a time remaining for the cassette to function, a dosing amount, or any combination thereof.

Yet in still further aspects, the user interface further comprises a portion comprising a control panel. In still further aspects, the control panel comprises an image representing the portion of the user interface that shows the status of the one or more receptacles and/or the status of the cassette inserted into the one or more receptacles.

Additional advantages will be set forth in part in the description that follows and in part will be evident from the description or can be learned by practice of the aspects described below. The advantages described below will be realized and attained by means of the chemical compositions, methods, and combinations thereof, particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects described below.

FIG. 1 depicts an exemplary apparatus according to one aspect.

FIG. 2 depicts an exemplary apparatus according to one aspect.

FIG. 3 depicts an exemplary apparatus according to one aspect.

FIGS. 4A-4B depict an exemplary apparatus according to one aspect.

FIG. 5 depicts an exemplary apparatus according to one aspect.

FIG. 6 depicts an exemplary apparatus according to one aspect.

FIG. 7 depicts an exemplary apparatus according to one aspect.

FIG. 8 depicts an exemplary apparatus according to one aspect.

FIG. 9 depicts an exemplary apparatus according to one aspect.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### DEFINITIONS

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise. Thus, for example, a reference to “a single-use unit” includes not only one but also two or more such units, and a reference to “an apparatus” includes not only one but also two or more such apparatuses and the like.

Throughout the description and claims of this specification, the word “comprise” and other forms of the word, such as “comprising” and “comprises,” are open, non-limiting terms and mean “including but not limited to,” and are not intended to exclude, for example, other additives, segments, integers, or steps. Furthermore, it is to be understood that the terms “comprise,” “comprising,” and “comprises” as they relate to various aspects, elements, and features of the disclosed invention also include the more limited aspects of “consisting essentially of” and “consisting of.”

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art.

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The expressions “ambient temperature” and “room temperature” as used herein are understood in the art and refer generally to a temperature from 20 °C to 35 °C.

All disclosed values also include values that fall within ±10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase’ x, y, z, or greater’ should be interpreted to include the specific ranges of ‘x,’ ‘y,’ ‘z,’ ‘about x,’ ‘about y,’ and ‘about z’ as well as the ranges of ‘greater than x,’ greater than y,’ ‘greater than z,’ or ‘greater than about x,’ greater than about y,’ ‘greater than about z.’ In addition, the phrase” ‘x’ to ‘y’,” where ‘x’ and y’ are numerical values, also includes “about x’ to about y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given, and exemplary values are provided, it is understood that any ranges can be formed between any exemplary values within the broadest range. For example, if individual numbers 1, 2, 3, 4, 5, 6, 7, etc. are disclosed, then the ranges 1-7, 2-7, 3-7, 4-7, 5-7, 6-7, 1-6, 1-5, 1-4, 1-3, 1-2, 2-6, 2-5, etc. are also disclosed.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “and/or” includes any and all combinations of one or more of the associated listed items.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can, in some aspects, refer to at least 90 %, at least 95 %, at least 99 %, or100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then 1 % by weight, e.g., less than 0.5 % by weight, less than 0.1 % by weight, less than 0.05 % by weight, or less than 0.01 % by weight of the stated material, based on the total weight of the composition.

As used herein, “treating” and “treatment” generally refer to obtaining a desired pharmacological or physiological effect. The effect can be but does not necessarily have to be prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and preventive treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

Some aspects described herein relate to methods. It should be understood that such methods can be computer-implemented. That is, where the method or other events are described herein, it should be understood that they may be performed by a computing device having a processor and a memory. Memory of a computing device is also referred to as a non-transitory computer‐readable medium, which can include instructions or computer code for performing various computer‐implemented operations. The computer‐readable medium (or processor‐readable medium) is non‐transitory in the sense that it does not include transitory propagating signals per se (e.g., a propagating electromagnetic wave carrying information on a transmission medium such as space or a cable). The media and computer code (also referred to as code) may be those designed and constructed for a specific purpose or purpose. Examples of non‐transitory computer‐readable media include but are not limited to magnetic storage media such as hard disks, floppy disks, and magnetic tape; optical storage media such as Compact Disc/Digital Video Discs (CD/DVDs), Compact Disc‐Read Only Memories (CD‐ROMs), and holographic devices; magneto‐optical storage media such as optical disks; carrier wave signal processing modules, Read‐Only Memory (ROM), Random‐Access Memory (RAM) and/or the like. One or more processors can be communicatively coupled to the memory and operable to execute the code stored on the non-transitory processor-readable medium. Examples of processors include general purpose processors (e.g., CPUs), Graphical Processing Units, Field Programmable Gate Arrays (FPGAs), Application Specific Integrated Circuits (ASICs), Digital Signal Processor (DSPs), Programmable Logic Devices (PLDs), and the like. Examples of computer code include, but are not limited to, micro‐code or micro‐instructions, machine instructions, such as those produced by a compiler, code used to produce a web service, and files containing higher‐level instructions that are executed by a computer using an interpreter. For example, aspects may be implemented using imperative programming languages (e.g., C, Fortran, etc.), functional programming languages (Haskell, Erlang, etc.), logical programming languages (e.g., Prolog), object‐oriented programming languages (e.g., Java, C++, etc.) or other suitable programming languages and/or development tools. Additional examples of computer code include, but are not limited to, control signals, encrypted code, and compressed code.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein.

### APPARATUS

Various systems and devices for generating nitric oxide (NO) are disclosed herein. Generally, NO is inhaled or otherwise delivered to a patient’s lungs. Since NO is inhaled, much higher local doses can be achieved without concomitant vasodilation of the other blood vessels in the body. Accordingly, NO gas having a concentration of 0.1 ppm to 1000 ppm (e.g., 0.1, 0.5, 1, 5, 10, 40, 80, 100, 150, 200, 250, 300, 350, 400, 450, 500, 550, 600, 650, 700, 750, 800, 850, 900, 950, or 1000 ppm) may be delivered to a patient. In still further aspects, the NO gas can have a concentration that falls between any two foregoing values or within a range formed by any two foregoing values. For example, NO gas can have a concentration of 0.1 ppm to 1000 ppm, 0.1 ppm to 900 ppm, 0.1 ppm to 800 ppm, 0.1 ppm to 700 ppm, 0.1 ppm to 600 ppm, 0.1 ppm to 500 ppm, 0.1 ppm to 400 ppm, 0.1 ppm to 300 ppm, 0.1 ppm to 200 ppm, 0.1 ppm to 100 ppm, 0.1 ppm to 80 ppm, 0.1 ppm to 50 ppm, 0.1 ppm to 20 ppm, 0.1 ppm to 10 ppm, 0.1 ppm to 5 ppm, 0.1 ppm to 1 ppm, 0.1 ppm to 0.5 ppm, 0.5 ppm to 1000 ppm, 1 ppm to 1000 ppm, 5 ppm to 1000 ppm, 10 ppm to 1000 ppm, 20 ppm to 1000 ppm, 50 ppm to 1000 ppm, 80 ppm to 1000 ppm, 100 ppm to 1000 ppm, 200 ppm to 1000 ppm, 300 ppm to 1000 ppm, 400 ppm to 1000 ppm, 500 ppm to 1000 ppm, 60 ppm to 1000 ppm, 700 ppm to 1000 ppm, or 800 ppm to 1000 ppm. Yet in other aspects, the NO concentration can be greater than 1000 ppm, greater than 1200 ppm, greater than 1500 ppm, greater than 1750 ppm, or even equal to or greater than 2000 ppm.

Accordingly, high doses of NO may be used to prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma, status asthmaticus, or hypoxia. NO can also be used to treat chronic pulmonary hypertension, bronchopulmonary dysplasia, chronic pulmonary thromboembolism, idiopathic pulmonary hypertension, primary pulmonary hypertension, or chronic hypoxia.

Currently, approved devices and methods for delivering inhaled NO gas require complex and heavy equipment. NO gas is stored in heavy gas bottles with nitrogen and no traces of oxygen. NO gas is mixed with air or oxygen with specialized injectors and complex ventilators, and the mixing process is monitored with equipment having sensitive microprocessors and electronics. All this equipment is required in order to ensure that NO is not oxidized into nitrogen dioxide (NO₂) during the mixing process since NO₂ is highly toxic. However, this equipment is not conducive to use in a non-medical facility setting since the size, cost, complexity, and safety issues restrict the operation of this equipment to highly-trained professionals in a medical facility.

Some of the devices for forming nitric oxide from NO₂ are disclosed in U.S. Patent Nos. 8,607,785, 8,944,049, 9,604,028, 10,926,054, 11,744,978, the contents of which are incorporated herein in their whole entirety.

In contrast, the devices and systems disclosed herein do not require the storage of nitric oxide in heavy gas bottles. The devices disclosed herein allow the formation of nitric oxide from nitrogen dioxide on demand, when a predetermined amount of nitric oxide is needed to be delivered to a patient.

Disclosed herein is an apparatus comprising: one or more receptacles. In such aspects, each of the receptacles is configured to receive a cassette comprising a nitric oxide-forming media and a user interface. In still further aspects, at least a portion of the user interface is configured to indicate: (a) a status of the one or more receptacles; and/or (b) a status of the cassette if inserted into the one or more receptacles.

It is understood that the nitric oxide-forming media can be any media known in the art and suitable for the use in the disclosed herein apparatus. In still further aspects, the nitric oxide-forming media can be media described in detailed in U.S. Provisional Application No. 63,573,175 and No. 63,728,392, the contents of which are incorporated herein in their full entirety.

In still further aspects, the at least a portion of the user interface further comprises one or more elements serving as an indicator of the status of the receptacle. Yet in still further aspects, the at least a portion of the user interface further comprises one or more elements serving as an indicator of the status of the cassette.

In still further aspects, the one or more elements are configured to assist with insertion in and ejection from the one or more receptacles of the cassette. In certain aspects, at least one of the one or more elements is an insertion/ejection element. In such exemplary and unlimiting aspects, the insertion/ejection element can be either an insertion element or ejection element, or both. In certain aspects, the insertion/ejection element can be an ejection button. In still further aspects, the insertion/ejection element can be mechanical. Yet, in still further aspects, the insertion/ejection element can be electronic. In certain aspects, the ejection button can have different lighting to indicate readiness. Yet in still further aspects, the ejection button can have a flashing capability to indicate the status of the receptacle and/or cassette. In yet still further aspects, the ejection button is configured to provide a sound aimed at alerting the user.

In still further aspects, the insertion/ejection element can indicate whether the receptacle is empty or whether the receptacle is occupied.

In still further aspects, the one or more elements can indicate one or more statuses of the cassette and/or the system. For example, and without limitations, the statuses of cassettes can include the following – inserted, locked, activated, warming, pressurizing, ready for dosing, actively dosing, dosing paused, dosing transitioning, dosing complete, cassette in need of replacement, cassette full, cassette empty, cassette near empty, cassette failure, cassette error, cassette not appropriately inserted, cassette ready for ejection.

In certain aspects, and without limitations, a portion or all of these states can be indicated by, for example, color variations of lights, type of lights, segmentation of lights, lights on vs off, brightness of light, and flashing of lights. In yet other aspects, the states can also be indicated by a sound.

In certain aspects, the one or more elements can comprise at least one element positioned anywhere at the at least a portion of the user interface and is configured to indicate the status of the cassette. For example, and without limitations, the at least one element is positioned around a receptacle opening. While in other exemplary aspects, the at least one element partially or fully encircles the one or more receptacles.

FIG. 1 shows apparatus 100 in one aspect of the present disclosure. In this exemplary aspect, receptacle 102 receives a cassette 1, and receptacle 108 receives a cassette 2. Each of the receptacles can have a lighted border 106 and 112. The lighted border 106 can have the same color as the lighted border 112. In other aspects, these two borders can have different colors. It can be seen here that the lighted borders 105 and 112 fully encircle the receptacle opening. However, it is understood that this aspect is only exemplary and unlimiting. When cassette 1 is depleted, border 106 will change to the color known to the user to indicate that the cassette is depleted. Further, when the apparatus is switched to cassette 2, border 112 will indicate to the user that the cassette is either getting ready to be operational or is in a stage of active dosing. The ejection buttons are shown as 104 and 110 in FIG. 1.

It is understood that the lighted border can be formed by any method known in the art. For example, the light borders can be formed by a multi-colored LED that can provide many different colors or can be another light source that has a number of fixed states or colors. In some embodiments, the lighting is white, and the surface color may be such that the white can be easily seen and provide a binary status, like “on,” “off,” “dosing,” or “not dosing.” In still further aspects, as indicated above, these lighted borders can also provide a sound or have wording or numbers shown within the border.

In still further aspects, the one or more elements can be illuminated by one or more colors, each indicating a different status of the receptacle and/or cassette. In still further aspects, the one or more elements comprise a word and/or number indication of the receptacle status and/or cassette status.

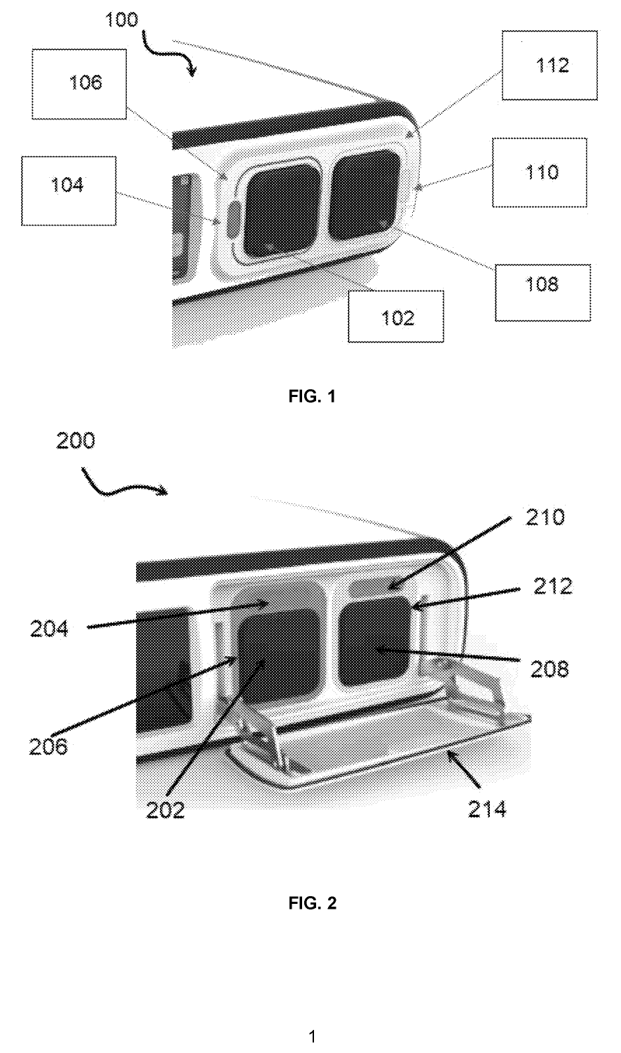

FIGS. 2-9 show additional exemplary and unlimiting apparatuses according to other aspects of the disclosure. In FIG. 2, the lighted areas 206 and 212 around the cassettes positioned in receptacles 202 and 208, which indicate their status, are broader and envelope ejection buttons 204 and/or 210 (manual or electronic). In certain aspects, at least a portion of the user interface indicating the status of the receptacle and/or cassette comprises a covering 214 configured to protect at least a portion of the user interface from damage. In such exemplary and unlimiting aspects, the covering is configured to be opened or closed. Yet in still further aspects, the covering can be transparent. Yet, in other aspects, the covering can be opaque.

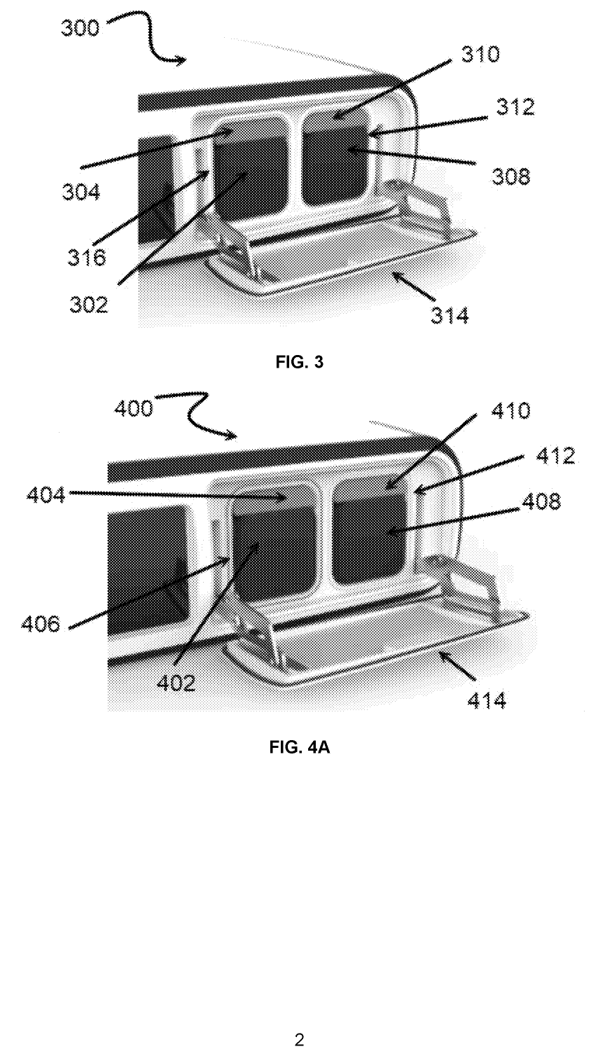

FIG. 3 shows apparatus 300 with mechanical buttons 304 and 310 above the receptacles 302 and 308 containing the cassettes and white lighted rings 306 and 312 that surround the receptacles. This white light may be solid or segmented and can also flash to indicate cassette status. It is understood that the ring can also be of any other color that is suitable for the desired application and not limited to white. In certain aspects, door (or covering) 314 can also be present to protect the receptacles. The door may be either opaque or transparent. In some aspects, it is transparent such that the lighting can be observed through the door when it is closed. In yet other aspects, the user would need to open the door in order to enable viewing of the status indicators.

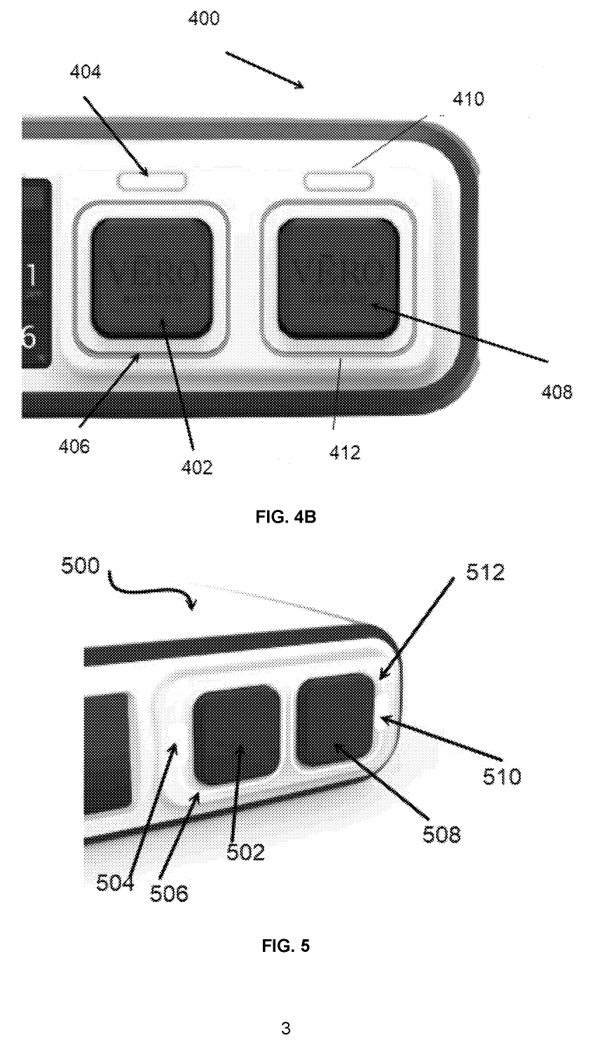

FIGS. 4A-4B show an exemplary apparatus 400 where colored light rings 406 and 412 substantially fully surround the receptacles 402 and 408 to indicate status. Ejection buttons 404 and 410 are above the receptacles and can be lighted or not lighted, manual or electronic. In FIG. 4A, light rings 406 and 412 encircled buttons 404 and 410, while in FIG. 4B, buttons 404 and 410 are positioned above the light rings 406 and 412.

FIG. 5 shows an exemplary apparatus 500 where lighted electronic ejection buttons 504 and 510 are to the left of the left receptacle 504 and the right of the right receptacle 508. These are contiguous with lighted rings 506 and 512 that surround the receptacles to indicate the status of the cassette and/or apparatus. For example, the lights can be white (but they also can have any other color) and may be on or off, flashing or solid, flashing at different speeds or rates.

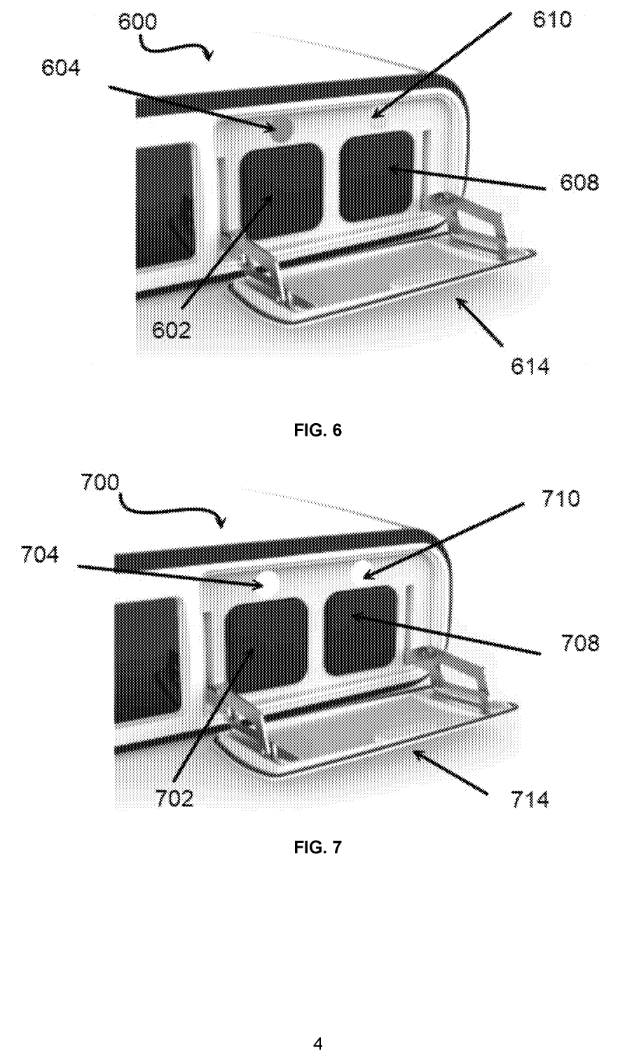

FIG. 6 shows an exemplary apparatus 600 whereas the status of the cassettes is displayed through distinctly lighted buttons 604 and 610. These buttons can have, for example, multi-colored capability and serve not only as status indicators for nitric oxide dosing and cassette status but also as an ejection button for the cassette.

FIG. 7 shows an exemplary apparatus 700 where white lights 704 and 710 (instead of multi-color shown in FIG. 6) indicate nitric oxide cassette status.

Yet in still further aspects, the one or more elements are in communication with the cassette and are positioned such that it is visually observable by a user.

In certain aspects, the one or more elements comprise a plurality of segments, wherein a sequence of the plurality of segments is configured to indicate the status of the cassette. In such exemplary and unlimiting aspects, the sequence and a number of segments indicate an amount of nitric oxide dosing remaining in the cassette, a time remaining for the cassette to function, a dosing amount, or any combination thereof. In still further aspects, the sequence and/or a number of elements are illuminated according to the status of the cassette.

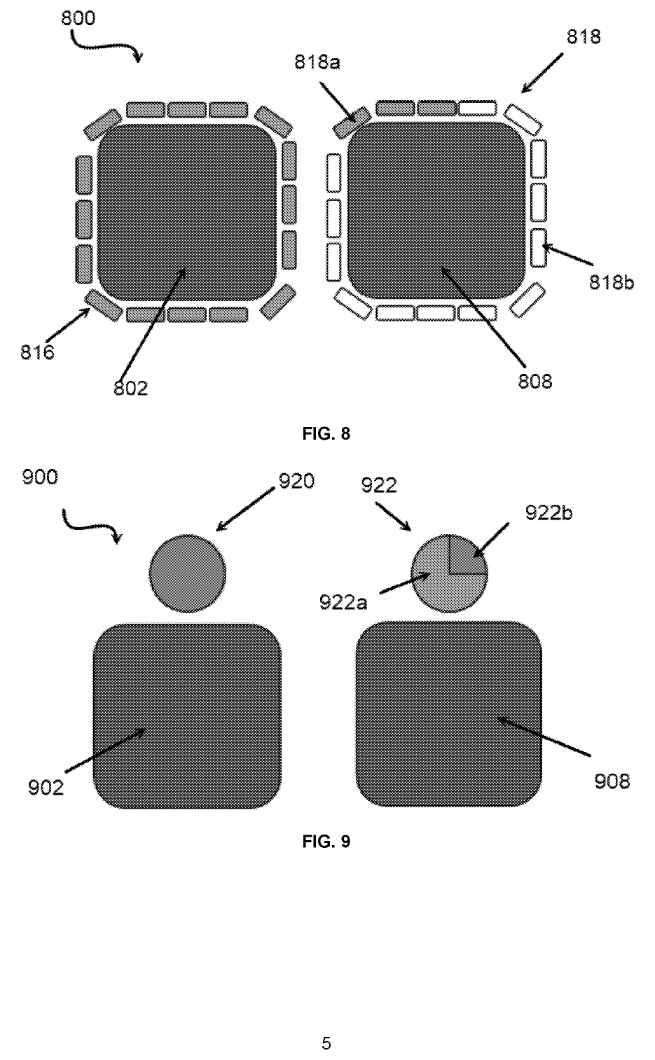

Such exemplary aspects are shown in FIG. 8. FIG. 8 shows apparatus 800, where the lighted borders 816 and 818 around the cassettes or receptacles 802 and 808 can be segmented to indicate a level. For example, the segments can show an indication of the amount of dosing time left, the amount of chemicals left, or the amount of power left. In such exemplary and unlimiting aspects, the lighted segments can begin to be turned off around the ring as the chemical is depleted until no lights are on, which indicates empty. For example, the darker segments shown as 818a can show that it is still in operation status, while the clear or white segment 818b shows the level of the cassette that was already spent. The segments may also form a continuous border around the cassette receptacle such that lighting of a given number of segments provides a continuous lighting of a portion of the border.

FIG. 9 shows an apparatus 900 where a status indication approach involving circles 920 and 922 could also be a lighted electronic button. Different amounts of the circle (922a and 922b) can be filled, and the color of the filling can also be adjusted. In this way, like a pie chart, the lighting can indicate the status of the cassette and an amount of time or other quantity like nitric oxide dose remaining. These lights can additionally flash to indicate status. Yet in still further aspects the one or more elements can be provided a constant illumination.

In still further aspects, the user interface can further comprise a portion comprising a control panel. In such aspects, for example, the control panel can also comprise an image representing the portion of the user interface that shows the status of the one or more receptacles and/or the status of the cassette inserted into the one or more receptacles. In still further aspects, the control panel can be at least partially a touch screen.

While various aspects have been described above, it should be understood that they have been presented by way of example only and not limitation. Furthermore, although various aspects have been described as having particular features and/or combinations of components, other aspects possibly have a combination of any features and/or components from any of the aspects where appropriate, as well as additional features and/or components.

Where methods described above indicate certain events occurring in a certain order, the ordering of certain events may be modified. Additionally, certain of the events may be performed concurrently in a parallel process, when possible, as well as performed sequentially as described above. Although various aspects have been described as having particular features and/or combinations of components, other aspects possibly have a combination of any features and/or components from any of the aspects where appropriate.

### EXEMPLARY ASPECTS

Example 1. An apparatus comprising: one or more receptacles, each configured to receive a cassette comprising a nitric oxide-forming media; and a user interface, wherein at least a portion of the user interface is configured to indicate: (a) a status of the one or more receptacles; and/or (b) a status of the cassette if inserted into the one or more receptacle.

Example 2. The apparatus of any one of the examples herein, particularly Example 1, wherein the at least a portion of the user interface further comprises one or more elements serving as an indicator of the status of the receptacle and/or the status of the cassette.

Example 3. The apparatus of any one of the examples herein, particularly Example 2, wherein the one or more elements are in communication with one or more receptacles and/or cassettes.

Example 4. The apparatus of any one of the examples herein, particularly Example 2 or 3, wherein the one or more elements are configured to assist with insertion in and ejection from the one or more receptacles of the cassette.

Example 5. The apparatus of any one of the examples herein, particularly Example 4, wherein at least one of the one or more elements is an insertion/ejection element.

Example 6. The apparatus of any one of the examples herein, particularly Example 5, wherein the insertion/ejection element is configured to indicate that the receptacle is empty or occupied.

Example7. The apparatus of any one of the examples herein, particularly Examples 4-6, wherein the one or more elements comprises at least one element positioned anywhere at the at least a portion of the user interface and is configured to indicate the status of the cassette.

Example 8. The apparatus of any one of the examples herein, particularly Example 7, wherein the at least one element is positioned around a receptacle opening.

Example 9. The apparatus of any one of the examples herein, particularly Example 7 or8, wherein the at least one element partially or fully encircles the one or more receptacles.

Example 10. The apparatus of any one of the examples herein, particularly Examples 4-9, wherein the one or more elements indicate one or more of the statuses of the cassette: inserted, locked, activated, warming, pressuring, ready for dosing, actively dosing, dosing paused, dosing transitioning, dosing complete, cassette in need of replacement, cassette full, cassette empty, cassette near empty., cassette dose remaining, cassette time remaining, cassette failure, cassette error, cassette is not inserted property, cassette ready for ejection, or any combination thereof.

Example 11. The apparatus of any one of the examples herein, particularly Examples 5-10, wherein the insertion/ejection element is mechanical or electronic.

Example 12. The apparatus of any one of the examples herein, particularly Examples 2-11, wherein the one or more elements are illuminated by one or more colors, each indicating a different status of the receptacle and/or cassette.

Example 13. The apparatus of any one of the examples herein, particularly Examples 2-12, wherein the one or more elements comprise a word and/or number indication of the receptacle status and/or cassette status.

Example 14. The apparatus of any one of the examples herein, particularly Examples 2-13, wherein the one or more elements are in communication with the cassette and are positioned such that it is visually observable by a user.

Example 15. The apparatus of any one of the examples herein, particularly Examples 2-14, wherein the one or more elements comprise a plurality of segments, wherein a sequence of the plurality of segments is configured to indicate the status of the cassette.

Example 16. The apparatus of any one of the examples herein, particularly Example 15, wherein the sequence and a number of segments indicate an amount of nitric oxide dosing remaining in the cassette, a time remaining for the cassette to function, dosing amount, or any combination thereof.

Example 17. The apparatus of any one of the examples herein, particularly Examples 15 or 16, wherein the sequence and/or a number of elements are illuminated according to the status of the cassette.

Example 18. The apparatus of any one of the examples herein, particularly Examples 2-17, wherein one or more elements are flashing or provide a constant illumination.

Example 19. The apparatus of any one of the examples herein, particularly Examples 1-18, wherein at least a portion of the user interface indicating the status of the receptacle and/or cassette comprises a covering configured to protect at least a portion of the user interface from damage.

Example 20. The apparatus of any one of the examples herein, particularly Example 19, wherein the covering is configured to be opened or closed.

Example 21. The apparatus of any one of the examples herein, particularly Example 19 or 20, wherein the covering is transparent.

Example 22. The apparatus of any one of the examples herein, particularly Examples 1-21, wherein the user interface further comprises a portion comprising a control panel.

Example 23. The apparatus of any one of the examples herein, particularly Examples 1 - 22, wherein the control panel comprises an image representing the portion of the user interface that shows the status of the one or more receptacles and/or the status of the cassette inserted into the one or more receptacles.

Example 24. The apparatus of any one of the examples herein, particularly Example 23, wherein the control panel is at least partially a touch screen.

What is claimed is:

1. An apparatus comprising:
one or more receptacles, each configured to receive a cassette comprising a nitric oxide-forming media; and
a user interface, wherein at least a portion of the user interface is configured to indicate:
a) a status of the one or more receptacles; and/orb) a status of the cassette if inserted into the one or more receptacles.

2. The apparatus of claim 1, wherein the at least a portion of the user interface further comprises one or more elements serving as an indicator of the status of the receptacle and/or the status of the cassette.

3. The apparatus of claim 2, wherein the one or more elements are in communication with one or more receptacles and/or cassettes.

4. The apparatus of claim 2 , wherein the one or more elements are configured to assist with insertion in and ejection from the one or more receptacles of the cassette.

5. The apparatus of claim 4, wherein at least one of the one or more elements is an insertion/ejection element.

6. The apparatus of claim 5, wherein the insertion/ejection element is configured to indicate that the receptacle is empty or occupied.

7. The apparatus of claim 4, wherein the one or more elements comprises at least one element positioned anywhere at the at least a portion of the user interface and is configured to indicate the status of the cassette.

8. The apparatus of claim 7, wherein the at least one element is positioned around a receptacle opening and/or wherein the at least one element partially or fully encircles the one or more receptacles.

9. The apparatus of claim 4, wherein the one or more elements indicates one or more of the statuses of the cassette: inserted, locked, activated, warming, pressurizing, ready for dosing, actively dosing, dosing paused, dosing transitioning, dosing complete, cassette in need of replacement, cassette full, cassette empty, cassette near empty, cassette dose remaining, cassette time remaining, cassette failure, cassette error, cassette is not inserted property, cassette ready for ejection, or any combination thereof.

10. The apparatus of claim 2, wherein the one or more elements are illuminated by one or more colors, each indicating a different status of the receptacle and/or cassette and /or wherein the one or more elements comprise a word and/or number indication of the receptacle status and/or cassette status.

11. The apparatus of claim 2, wherein the one or more elements are in communication with the cassette and are positioned such that it is visually observable by a user.

12. The apparatus of claim 2, wherein the one or more elements comprise a plurality of segments, wherein a sequence of the plurality of segments is configured to indicate the status of the cassette.

13. The apparatus of claim 12, wherein the sequence and a number of segments indicate an amount of nitric oxide dosing remaining in the cassette, a time remaining for the cassette to function, a dosing amount, or any combination thereof and/or wherein the sequence and/or a number of elements are illuminated according to the status of the cassette.

14. The apparatus of claim 2, wherein one or more elements are flashing or provide a constant illumination.

15. The apparatus of claim 1, wherein at least a portion of the user interface indicating the status of the receptacle and/or cassette comprises a covering configured to protect at least a portion of the user interface from damage.

16. The apparatus of claim 15, wherein the covering is configured to be opened or closed.

17. The apparatus of claim 15, wherein the covering is transparent.

18. The apparatus of claim 1, wherein the user interface further comprises a portion comprising a control panel.

19. The apparatus of claim 18, wherein the control panel comprises an image representing the portion of the user interface that shows the status of the one or more receptacles and/or the status of the cassette inserted into the one or more receptacles.

20. The apparatus of claim 19, wherein the control panel is at least partially a touch screen.
